(19) United States
(12) Patent Application Publication
(10) Pub. No.: US 2025/0124741 A1
(43) Pub. Date: Apr. 17, 2025

(54) ACTION RECOGNITION DEVICE, ACTION RECOGNITION METHOD, AND NON-TRANSITORY COMPUTER READABLE RECORDING MEDIUM

(71) Applicant: Panasonic Intellectual Property Corporation of America, Torrance, CA (US)

(72) Inventors: Nobuhiko WAKAI, Tokyo (JP); Shigehiro IIDA, Tokyo (JP)

(73) Assignee: Panasonic Intellectual Property Corporation of America, Torrance, CA (US)

(21) Appl. No.: 18/999,988

(22) Filed: Dec. 23, 2024

Related U.S. Application Data
(63) Continuation of application No. PCT/JP2022/048524, filed on Dec. 28, 2022.

(30) Foreign Application Priority Data
Jul. 6, 2022 (JP) 2022-109151

(51) Int. Cl. G06V 40/20; G06V 10/32; G06V 10/62; G06V 10/75

(52) U.S. Cl. CPC G06V 40/25; G06V 10/32; G06V 10/62; G06V 10/751

(57) ABSTRACT

An action recognition device performs: estimating node coordinates of a user from an image; calculating, on the basis of the estimated node coordinates, time-series feature points each indicating a feature point of a trunk of the user in time series; and a deciding a change in the walk of the user by comparing input time-series feature points being the calculated time-series feature points with time-series feature points being reference time-series feature points each to be a reference.

### FIELD OF INVENTION

The present disclosure relates to a technology of recognizing an action of a user from an image.

### BACKGROUND ART

For instance, Patent Literature 1 and Patent Literature 2 have been known to each disclose a technology of recognizing an action of a user by using an image, such as a still image and a video image. Patent Literature 1 has an object of determining a fatigue degree of a person, and discloses a technology of extracting time-series data of a node of a person on the basis of a video image captured by photographing a target user and determining the fatigue degree on the basis of an arm swinging state and a foot fluctuation of the user. Patent Literature 2 has an object of determining a walking state and an age of a walking person, and discloses a technology of detecting time-series data about a specific portion of a person on the basis of a video image captured by photographing a target user, and calculating a walking state of the person and a classification of a walking velocity of the person.

A camera to be arranged in a residence may not be at a position suitable for sensing due to restrictions including a room layout and a room dimension, and thus occasionally fails to capture a front image in a walk at a distance within five to ten meters that is generally used in an analysis of a walking motion.

Unfortunately, the aforementioned conventional action recognition technologies do not consider such a failure at photographing a walking user from the front thereof, and thus needs further improvement in deciding a change in the walk of the user.
Patent Literature 1: Japanese Patent Publication No. 6873344
Patent Literature 2: Japanese Patent Publication No. 3655618

### SUMMARY OF THE INVENTION

The present disclosure has been accomplished to solve the drawbacks described above, and has an object of providing a technology of deciding a change in a walk of a user with a high accuracy even in a failure at photographing the waking user from the front thereof.

An action recognition device according to one aspect of the present disclosure is an action recognition device that recognizes a walk of a user. The action recognition device includes: an acquisition part that acquires an image; an estimation part that estimates node coordinates of the user from the image acquired by the acquisition part; a calculation part that calculates, on the basis of the node coordinates estimated by the estimation part, time-series feature points each indicating a feature point of a trunk of the user in time series; a storage part that stores time-series feature points being reference time-series feature points each to be a reference; and a decision part that decides a change in the walk of the user by comparing input time-series feature points being the time-series feature points calculated by the calculation part with the reference time-series feature points.

The present disclosure achieves decision on a change in a walk of a user with a high accuracy even in a failure at photographing the walking user from the front thereof.

### BRIEF DESCRIPTION OF THE DRAWINGS

FIG. 1 is a block diagram showing an example of a configuration of an action recognition system in a first embodiment of the present disclosure.

FIG. 2 is an illustration of an example of node information showing node coordinates estimated by an estimation part.

FIG. 3 is a flowchart showing an example of a process by an action recognition device in the first embodiment of the disclosure.

FIG. 4 is an illustration of an example of a feature point.

FIG. 5 is a graph showing an example of a polynomial trendline.

FIG. 6 is a flowchart showing details of step S9 in FIG. 3.

FIG. 7 is a graph explaining a way of deciding a change in a walk with a reference deviation of an error calculated in step S8.

FIG. 8 is a block diagram showing an example of a configuration of an action recognition system in a second embodiment of the present disclosure.

FIG. 9 is a flowchart showing an example of a process by an action recognition device in the second embodiment of the disclosure.

FIG. 10 is a block diagram showing an example of a configuration of an action recognition system in a third embodiment of the present disclosure.

FIG. 11 is a flowchart showing an example of a process by an action recognition device in the third embodiment of the disclosure.

FIG. 12 is a flowchart showing details of step S303 in FIG. 11.

### DETAILED DESCRIPTION

Knowledge forming the basis of the present disclosure

In recent years, a way of estimating a node of a person from an image and recognizing an action of a user on the basis of the estimated node has been known. The recognition way adopts an image captured by photographing a whole body of the user at a camera angle suitable for sensing, and images captured by photographing the user in a walking state for a long time. The recognition way further includes estimating a node via a deep neural network including a convolutional layer and a pooling layer to increase the accuracy.

Meanwhile, a technology of detecting a change in a walk of a user in a daily life has been expected in recent years to detect a physical decline. Unfortunately, in such a daily life, some obstacle may hide a part of the body of the user whose action is to be recognized or a part of the body may not be covered in an image. The conventional recognition way hence has a drawback of a failure at deciding a change in a walk of a user with a high accuracy at photographing the user in the action under an unfavorable photographing condition. In particular, this drawback is likely to be seen in a residence having a restriction on an arrangement position for the camera.

This disclosure has been conceived in consideration of the drawbacks described above, and has an object of providing a technology of deciding a change in a walk of a user to be recognized with a high accuracy even from an image not covering a whole body of the user.
(1) An action recognition device according to one aspect of the present disclosure is an action recognition device that recognizes a walk of a user. The action recognition device includes: an acquisition part that acquires an image; an estimation part that estimates node coordinates of the user from the image acquired by the acquisition part; a calculation part that calculates, on the basis of the node coordinates estimated by the estimation part, time-series feature points each indicating a feature point of a trunk of the user in time series; a storage part that stores time-series feature points being reference time-series feature points each to be a reference; and a decision part that decides a change in the walk of the user by comparing input time-series feature points being the time-series feature points calculated by the calculation part with the reference time-series feature points.

In this configuration, a change in the walk of the user is decided by comparing the input time-series feature points with the reference time-series feature points. Here, each of the input time-series feature points and the reference time-series feature points constitutes a feature point of a trunk of the user. The feature point is generatable on the basis of information about an upper body of the user and does not significantly fluctuate depending on a camera angle. This configuration hence enables decision on a change in the walk of the user to be recognized with a high accuracy even from an image not covering a whole body of the user.
(2) In the action recognition device according to (1), the time-series feature points may be normalized in such a manner that a time required for the walk is a reference value.

In this configuration, each of the input time-series feature points and the reference time-series feature points is normalized in such a manner that a time required for the walk is a reference value. The configuration thus facilitates the comparison between the input time-series feature points and the reference time-series feature points.
(3) The action recognition device according to (1) or (2) above may further include a determination part that determines, on the basis of the time-series feature points calculated by the calculation part, whether the user walks straight. The decision part may decide a change in the walk of the user when the user is determined to walk straight.

This configuration executes the decision on the change in the walk of the user when the user walks straight while avoiding the execution of the decision on the change in the walk of the user when the user does not walk straight, and thus enables the decision on the change in the walk of the user with a high accuracy.
(4) The action recognition device according to any one of (1) to (3) above may further include a depth correction part that calculates correction factors to distances between the user shown in the image and a camera that captures the image of the user in time series. The decision part may decide a change in the walk of the user by normalizing the time series feature points by multiplying feature points constituting the time-series feature points by correction factors corresponding to the feature points respectively, and comparing the normalized time-series feature points with the reference time-series feature points which are normalized.

This configuration enables comparison between the input time-series feature points and the reference time-series feature points after removing a change in a size of the user shown in the image in accordance with a distance from the camera to the user.
(5) In the action recognition device according to any one of (1) to (4) above, the calculation part may calculate respective polynomial approximate curves of X-coordinates and Y-coordinates of the time-series feature points on the basis of image coordinates of the calculated time-series feature points, and correct respective values of X-coordinates and Y-coordinates of feature points constituting the time-series feature points by using the calculated polynomial approximate curves of the X-coordinates and the Y-coordinates.

In this configuration, the estimated time-series node coordinates may include an error, but the polynomial approximate curves of X-coordinates and Y-coordinates of the time-series feature points are used to correct a value on each of an X-coordinate and a Y-coordinate of each of the time-series feature points, so that the time-series feature points form a linear locus. The configuration thus enables decision on a change in the walk of the user with a high accuracy.
(6) In the action recognition device according to any one of (1) to (5) above, the reference time-series feature points may include a plurality of first reference time-series feature points, and the decision part may decide a change in the walk of the user by calculating an average time-series feature point by averaging the first reference time-series feature points, and comparing the average time-series feature point with the input time-series feature points.

In this configuration, an average time-series feature resulting from averaging the first reference time-series feature points is compared with each of the input time-series feature points, and thus, the configuration enables one-to-one comparison between the input time-series feature points and the first reference time-series feature points.
(7) In the action recognition device according to any one of (1) to (6), in a case where a period during which time-series feature points are continuously calculated is not shorter than a predetermined time, the calculation part may determine the feature points within the period as the input time-series feature points.

This configuration enables determination of time-series feature points formed of a series of feature points representing a walk as the input time-series feature points, and thus enables recognition of a change in the walk of the user with a high accuracy.
(8) In the action recognition device according to any one of (1) to (7) above, the reference time-series feature points may represent input time-series feature points calculated by the calculation part in past.

In this configuration, the input time-series feature points calculated in past serve as reference time-series feature points, and thus, the configuration facilitates collecting of the reference time-series feature points. Further, the past input time-series feature points and current input time-series feature points are compared with each other. The configuration hence enables decision on a change from a past walk with a high accuracy.
(9) In the action recognition device according to any one of (1) to (8) above, the reference time-series feature points and the input time-series feature points may belong to the same user.

This configuration enables decision on a change from a past walk of the user to a current walk of the user through comparison between the walks.
(10) The action recognition device according to any one of (1) to (9) above may further include an output part that outputs information indicating the change in the walk of the user decided by the decision part.

This configuration enables output of information indicating a decided change in the walk.
(11) An action recognition method according to another aspect of the disclosure is an action recognition method for an action recognition device that recognizes a walk of a user. The action recognition method includes: acquiring an image; estimating node coordinates of the user from the acquired image; calculating, on the basis of the estimated node coordinates, time-series feature points each indicating a feature point of a trunk of the user; and deciding a change in the walk of the user by comparing input time-series feature points being the calculated time-series feature points with time-series feature points being reference time-series feature points each to be a reference.

This configuration makes it possible to provide an action recognition method that exerts operational effects equivalent to those of the action recognition device described above.
(12) An action recognition program according to further another aspect of the disclosure is an action recognition program for causing a computer to execute an action recognition method for recognizing a walk of a user. The action recognition program includes causing a computer to execute: acquiring an image; estimating node coordinates of the user from the acquired image; calculating, on the basis of the estimated node coordinates, time-series feature points each indicating a feature point of a trunk of the user; and deciding a change in the walk of the user by comparing input time-series feature points being the calculated time-series feature points with time-series feature points being reference time-series feature points each to be a reference.

This configuration makes it possible to provide an action recognition method that exerts operational effects equivalent to those of the action recognition device described above.
(13) A recording medium according to still another aspect of the disclosure is a non-transitory computer readable recording medium for recording an action recognition program for causing a computer to execute an action recognition method for recognizing a walk of a user. The recording medium includes causing the computer to execute: acquiring an image; estimating node coordinates of the user from the acquired image; calculating, on the basis of the estimated node coordinates, time-series feature points each indicating a feature point of a trunk of the user; and deciding a change in the walk of the user by comparing input time-series feature points being the calculated time-series feature points with time-series feature points being reference time-series feature points each to be a reference.

This disclosure can be realized as an action recognition system caused to operate by the action recognition program as well. Additionally, it goes without saying that the computer program is distributable as a non-transitory computer readable storage medium like a CD-ROM, or distributable via a communication network like the Internet.

Each of the embodiments which will be described below represents a specific example of the disclosure. Numeric values, shapes, constituent elements, steps, and the order of the steps described below in each embodiment are mere examples, and thus should not be construed to delimit the disclosure. Moreover, constituent elements which are not recited in the independent claims each showing the broadest concept among the constituent elements in the embodiments are described as selectable constituent elements. The respective contents are combinable with each other in all the embodiments.

### First Embodiment

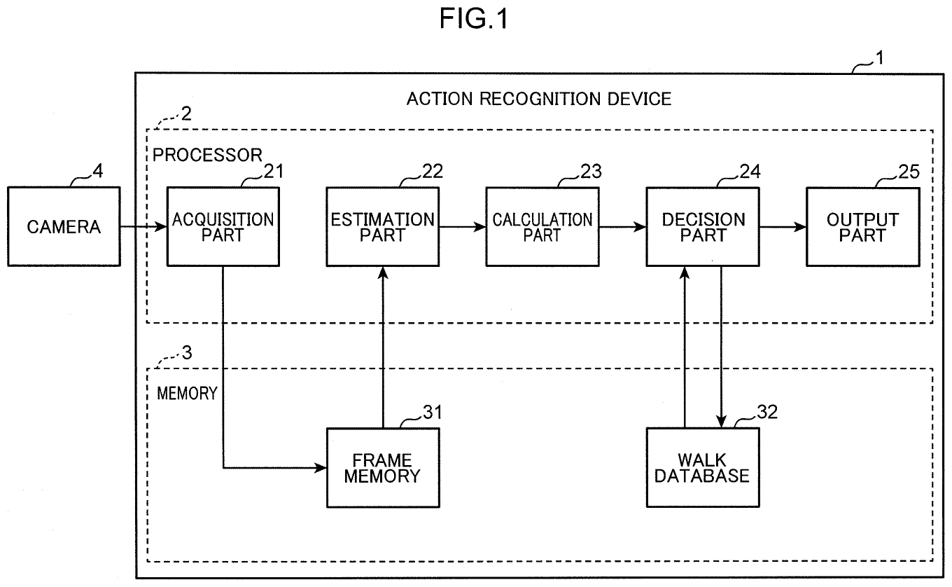

Hereinafter, an embodiment of the disclosure will be described with reference to the accompanying drawings. FIG. 1 is a block diagram showing an example of a configuration of an action recognition system in a first embodiment of the present disclosure. The action recognition system includes an action recognition device 1 and a camera 4. The camera 4 is a photographing device that captures an image of an action of a user (hereinafter, referred to as a “target user”) whose action is to be recognized. For instance, a fixed camera provided in a residence where the user lives corresponds to the camera, but is not particularly limited thereto. The camera 4 captures an image of the user at a predetermined frame rate, and inputs the captured image into the action recognition device 1 at a predetermined frame rate. The action recognition device 1 is aimed at deciding a change in a walk of the target user by using the image captured by the camera. The action recognition device 1 is composed of a computer including a processor 2, a memory 3, and an interface circuit (not shown).

The processor 2 includes hardware for deciding a change in the walk of the target user on the basis of the image, for example, a central processing unit. The processor 2 has an acquisition part 21, an estimation part 22, a calculation part 23, a decision part 24, and an output part 25. Each of the acquisition part 21 to the output part 25 may come into effect when the central processing unit executes an action recognition program, or may be established in the form of a dedicated hardware circuit, such as an application specific integrated circuit (ASIC).

The memory 3 is a storage device that records a walk of the target user acquired from the image, that is, includes a non-volatile storage device, for example, a flash memory, a hard disk drive, and a solid state drive. The interface circuit is an input and output mechanism for data, and includes, for example, a communication circuit.

The memory 3 has a frame memory 31 and a walk database 32. The frame memory 31 stores an image acquired by the acquisition part 21 from the camera 4. The walk database 32 is a database that stores input time-series feature points calculated from images of the target user in past as reference time-series feature points.

The acquisition part 21 acquires an image captured by the camera 4 and stores the acquired image in the frame memory 31. The frame memory 31 stores the image input from the acquisition part 21.

The estimation part 22 reads out an image from the frame memory 31 and estimates a plurality of node coordinates of the target user and reliability of each of the node coordinates on the basis of the read image. Each node coordinate represents a coordinate in an image coordinate system of a node of the target user detected from the image.

The reliability is a value indicating a certainty of estimation for each node coordinate and expresses the certainty of the estimated node coordinate with a probability. The certainty increases as a value of the reliability of the node coordinate increases. The reliability takes, for example, a value falling within a range from 0 to 1.

The estimation part 22 estimates each of the node coordinates and the reliability thereof by inputting the image into a learned model obtained through machine learning of a relation between the image and the node coordinate. An example of the learned model is a deep neural network. An example of the deep neural network is a convolutional neural network including a convolutional layer and a pooling layer. The estimation part 22 may include a learning model other than the deep neural network.

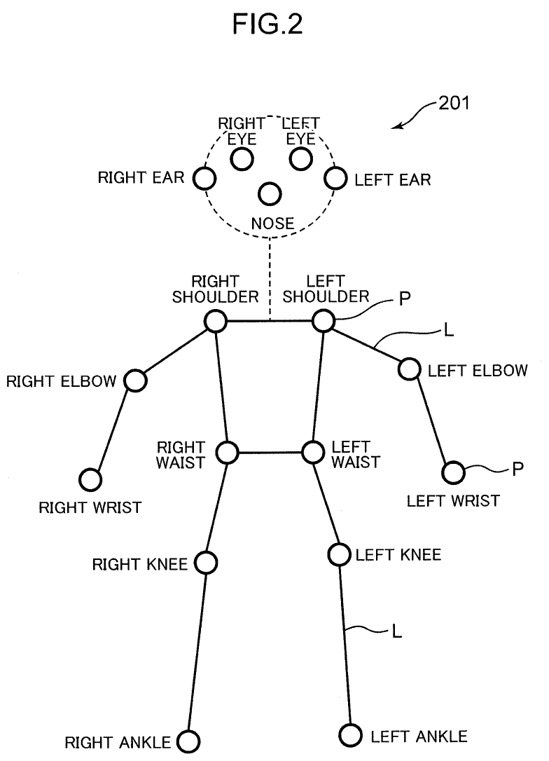

FIG. 2 is an illustration of an example of node information 201 showing node coordinates P estimated by the estimation part 22. In FIG. 2, dashed lines represent auxiliary lines respectively denoting an outline of a face and a location of a neck.

The node information 201 includes information about node coordinates P of one target user. The node information 201 shows, for example, seventeen node coordinates P respectively showing a left eye, a right eye, a left ear, a right ear, a nose, a left shoulder, a right shoulder, a left waist, a right waist, a left elbow, a right elbow, a left wrist, a right wrist, a left knee, a right knee, a left ankle, and a right ankle. The estimation part 22 is configured to estimate each of the node coordinates P. In the example shown in FIG. 2, the node information 201 shows the seventeen node coordinates P1 to P17, but this is just an example. The number of node coordinates P may be sixteen or smaller, or may be eighteen or larger. In this case, the learned model is configured to estimate a predetermined number of node coordinates P that is sixteen or smaller, or eighteen or larger. Furthermore, the node information 201 may show other nodes (e.g., nodes of a finger, a mouth, and other parts) in addition to the node coordinates P shown in FIG. 2.

The node information 201 further shows links L linking the node coordinates P to each other. Each of the node coordinates P is expressed by an X-coordinate being a horizontal component in the image and a Y-coordinate being a vertical component in the image. The node information 201 is expressed by an indicator uniquely specifying each node coordinate P, the node coordinate P, and reliability of the node coordinate P. For instance, the node information 201 is expressed in a dictionary format, such as {an indicator “right eye”: [X-coordinate, Y-coordinate, reliability], an indicator “left eye”: [X-coordinate, Y-coordinate, reliability], . . . , an indicator “left ankle”: [X-coordinate, Y-coordinate, reliability]}.

The calculation part 23 calculates, on the basis of the node coordinates P estimated by the estimation part 22, time-series feature points each indicating a feature point of a trunk of the target user in time series. Hereinafter, the time-series feature points are referred to as input time-series feature points. A feature point is a point in an image coordinate system calculated from one image.

The decision part 24 decides a change in the walk of the target user by comparing the input time-series feature points calculated by the calculation part 23 with the reference time-series feature points stored in the walk database 32. The decision will be described in detail later. The decision part 24 may express a change in an action by a Boolean value, that is, a true or false value. However, this is just an example, and the decision part 24 may calculate a numeric value indicating a difference between each input time-series feature point and each reference time-series feature point as a change in the walk of the target user. The Boolean value shows “true” in accordance with a change in an action and “false” in accordance with no change in the action.

The reference time-series feature points to be compared with the input time-series feature points represent input time-series feature points of the target user calculated in past by the calculation part 23, for example, calculated a half-year before. However, this is just an example, and the reference time-series feature points to be compared with the input time-series feature points may represent time-series feature points of another user who is at the same age as the target user, or may represent time-series feature points each indicating a reference walk stored in advance.

The output part 25 outputs information indicating the change in the walk of the target user decided by the decision part 24. Example cases each showing a change in a walk of a target user include a case where a walk of the target user photographed a half-year before differs from a walk of the target user currently photographed and a case where a difference is seen in the walk of the target user due to a physical decline attributed to aging or an injury. The output part 25 may send the information to a computer carried by the target user via an input and output interface, or may store the information in a storage device.

For instance, the action recognition device 1 may include an edge server provided in the residence, may include a smart speaker provided in the residence, or may include a cloud server. When the action recognition device 1 includes the edge server, the camera 4 and the action recognition device 1 are connected to each other via a local area network. When the action recognition device 1 includes the cloud server, the camera 4 and the action recognition device 1 are connected to each other via a wide area network like the internet. Here, a part of the constituent elements of the action recognition device 1 may be provided on the edge server, and the remaining part of the constituent elements may be provided on the cloud server.

The action recognition device 1 may not be necessarily achieved by a single computer device, but may be achieved by a decentralization system (not shown) including a terminal device and a server. In this case, the acquisition part 21, the frame memory 31, and the estimation part 22 may be provided in the terminal device, whereas the walk database 32, the calculation part 23, the decision part 24, and the output part 25 may be provided on the server. In this case, the constituent elements transfer data therebetween via a wide area network.

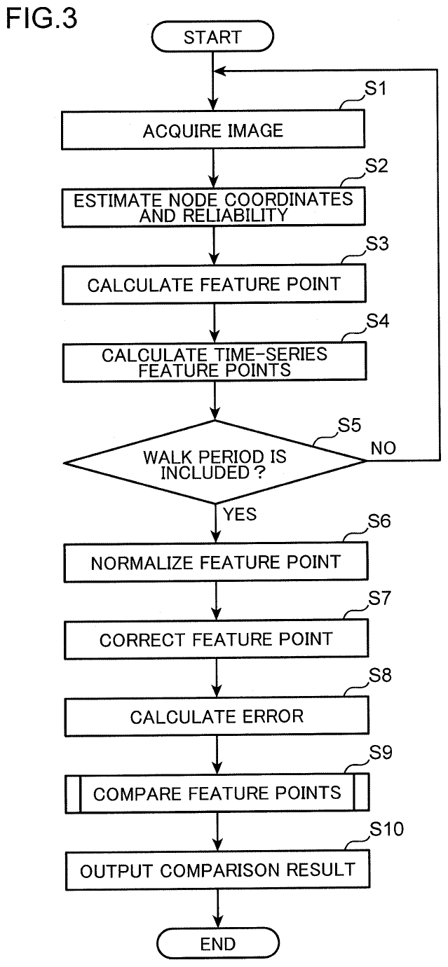

Heretofore, the configuration of the action recognition device 1 has been described. Next, a process by the action recognition device 1 will be described. FIG. 3 is a flowchart showing an example of the process by the action recognition device 1 in the first embodiment of the disclosure.

### Step S1

The acquisition part 21 acquires an image from the camera 4 and stores the acquired image in the frame memory 31. The acquisition part 21 may always acquire an image or may periodically acquire an image from the camera 4.

### Step S2

The estimation part 22 acquires a plurality of images in time series from the frame memory 31, and estimates a plurality of node coordinates P in each image and reliability of each of the node coordinates P by inputting the acquired images into a learned model one after another. In this manner, time-series data of the node coordinates P and the reliability are obtained. The estimation part 22 may trace the target user with the time-series data. For instance, the estimation part 22 may calculate gravity centers of bounding rectangles concerning the node coordinates P in respective continuous time-series images, and associate a bounding rectangle having the gravity center closest to the gravity center of another bounding rectangle with each other to determine that the bounding rectangles belong to the same user, resulting in tracing the target user. In this manner, even in a case where an image shows a plurality of target users, the configuration enables tracing of the target users individually. Alternatively, the estimation part 22 may associate bounding rectangles in adjacent images with each other by the Hungarian notation. In a case of a plurality of gravity centers respectively in adjacent images, a set of gravity centers at a shortest distance therebetween can be determined by the Hungarian notation.

Although an image is described to show one target user in the first embodiment, this is an example, and an image may show a plurality of target users. In this case, the action recognition device 1 may estimate node coordinates P and reliability of each of the node coordinate P for each of the target users individually, and decide a change in an action of each target user in accordance with an associated result of the estimation.

### Step S3

The calculation part 23 calculates a feature point of the trunk of the target user from the node coordinates P acquired in step S2. In the embodiment, feature points calculated from the node coordinates P on the basis of the node coordinates P and the reliability in time series are referred to as time-series feature points.

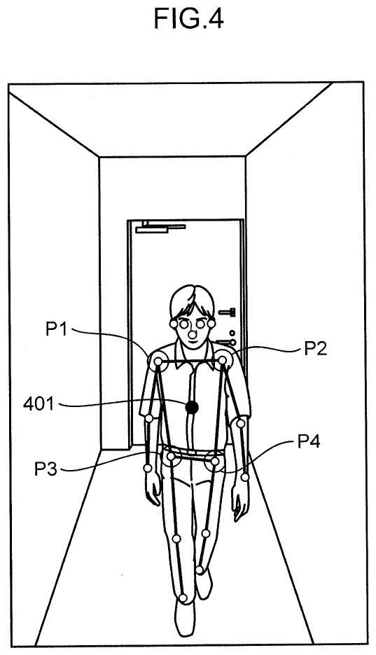

FIG. 4 is an illustration of an example of a feature point 401 calculated from an image captured by photographing a walking user. The feature point 401 represents a reference point of an upper body on an image coordinate and indicates a gravity center on node coordinates P of the trunk. The feature point 401 is a gravity center on four node coordinates P in total of the node coordinates P1, P2 of both the shoulders and the node coordinates P3, P4 of both the waists of the target user.

The calculation part 23 may exclude such a node coordinate P as not detected due to, for example, an obstacle and an orientation of the body from the calculation of the feature point 401 to increase an accuracy of estimating the feature point 401. In this case, the calculation part 23 may calculate the feature point 401 by using only a detected node coordinate P. The calculation part 23 may avoid calculating the feature point 401 at no detection of a node coordinate P which is necessary for the calculation of the feature point 401 from the acquired image. In this case, the calculation part 23 may record a missing value indicating no feature point 401 in place of the feature point 401. In this manner, the calculation part 23 can calculate a group of feature points 401 in a period excluding the missing value as input time-series feature points.

The calculation part 23 may calculate the feature point 401 by weighting and averaging the node coordinates P1 to P4 belonging to the trunk by the reliability of the node coordinates P1 to P4. The calculation part 23 may calculate the gravity center of a bounding rectangle including the node coordinates P1 to P4 as the feature point 401 in place of the gravity center on the node coordinates P1 to P4. The node coordinates P for use in calculation of the feature point 401 are not limited to the node coordinates P1 to P4, and other node coordinate P may be added. For instance, the calculation part 23 may calculate the feature point 401 by further using the node coordinates P of both the knees and both the elbows.

### Step S4

The calculation part 23 calculates time-series feature points by extracting, from among feature points 401 calculated in step S3, feature points 401 in a predetermined period from a current time to a certain past time. The predetermined period indicates, for example, ten seconds, but is not particularly limited thereto. The predetermined period may show a plurality of walks. In this case, the calculation part 23 may extract feature points 401 for each of the walks. Specifically, when a predetermined period includes a subperiod during which feature points 401 are continuously calculated for a period of a threshold or longer (e.g., for two seconds or longer), the calculation part 23 may define the subperiod as a walking period and calculate time-series data about the feature points 401 in the walk period to be input time-series feature points. The calculation part 23 may define a missing subperiod for which the missing value is recorded in step S3 as a boundary of the walk period. Thus, when the predetermined period includes the walk periods, input time-series feature points associated with each walk period are calculated. This leads to calculation of input time-series feature points for the walk period being a period of the threshold or longer and enables accurate specifying of the walk of the user. Specifically, in calculation of a plurality of input time-series feature points in a predetermined period in the embodiment, a change in the walk is decided for each of the input time-series feature points. An operator having viewed an image may manually designate extraction of a predetermined period and a walk period.

### Step S5

The calculation part 23 leads the process to step S6 when the predetermined period includes a walk period in step S5 (YES in step S5), and returns the process to step S1 when the walk period is not included (NO in step S5).

### Step S6

The calculation part 23 normalizes each of the input time-series feature points in such a manner that a time for each of the input time-series feature points calculated in step S4 is a reference value (e.g., 1). Although a time required for a motion may differ in a specific walk, the normalization allows a time scale for input time-series feature points and a time scale for reference time-series feature points to agree with each other, and thus facilitates comparison between the input and reference time-series feature points. For example, a walk is defined to come to a peak at the middle of a valid subperiod. In this example, a walk in four seconds for input time-series feature points comes to a peak at a position of two seconds, and a walk in six seconds for reference time-series feature points comes to a peak at a position of three seconds. The peak about the input time-series feature points does not agree with the peak about the reference time-series feature points. In this regard, the aforementioned normalization makes respective peaks about the input and reference time-series feature points reach a position of 0.5, and thus facilitates comparison between the input and reference time-series feature points.

The normalization is not limited thereto. For instance, the calculation part 23 may normalize an X-coordinate (a horizontal coordinate of an image) and a Y-coordinate (a vertical coordinate of the image) of each feature point so that a horizontal length and a vertical length of an image in each frame for each input time-series feature point have a reference value (e.g., “1”). This enables comparison between feature points 401 shown in images respectively captured by cameras having different solutions.

### Step S7

The calculation part 23 corrects each feature point included in the input time-series feature points. The node coordinates P estimated by the estimation part 22 include an estimation error, and thus, a locus of feature points 401 constituting the input time-series feature points on the image is not smooth. Therefore, the calculation part 23 approximates the time-series feature points by a polynomial to form a smooth movement locus of the feature points 401 constituting the input time-series feature points on the image.

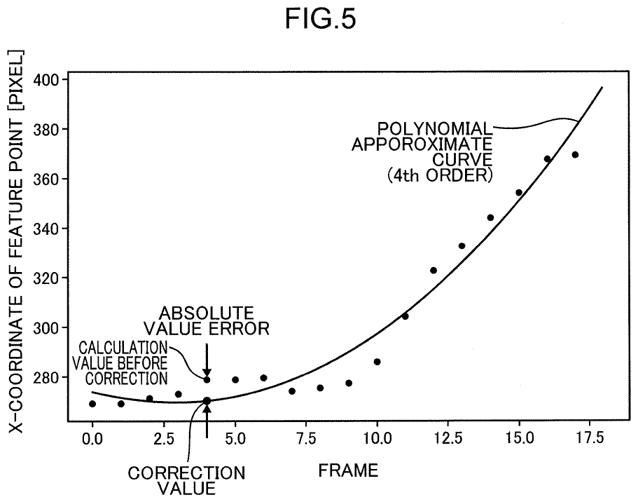

FIG. 5 is a graph showing an example of a polynomial trendline for correcting the input time-series feature points. In FIG. 5, the horizontal axis denotes a frame, and the vertical axis denotes an X-coordinate of a feature point.

First, the calculation part 23 calculates respective polynomial approximate curve for X-coordinates of the input time-series feature points on the basis of image coordinates of the calculated input time-series feature points. When plotting a value (v) on each of X-coordinates of input time-series feature points to a value (u) of a frame (time) associated with each of the input time-series feature points, the calculation part 23 fits the polynomial g with “v=(u)” to the input time-series feature points and calculates polynomial approximate curve. The N-th order of the polynomial g is, for example, “the 4^{th}” order.

The calculation part 23 corrects the X-coordinates of the input time-series feature points by using the calculated polynomial approximate curve for the X-coordinates. The calculation part 23 calculates a correction value of the feature point 401 on the X-coordinate by assigning a value of a calculated value u of the feature point before correction into the polynomial g.

Similarly, the calculation part 23 calculates a correction value of the Y-coordinate of the feature point. Specifically, the calculation part 23 calculates respective polynomial approximate curve for Y-coordinates of the input time-series feature points on the basis of the image coordinates of the calculated input time-series feature points. The calculation part 23 then corrects a value on each of the Y-coordinates of the feature points 401 in time series by using the calculated polynomial approximate curve for the Y-coordinates.

### Step S8

The calculation part 23 calculates an error between the polynomial g and the X-coordinate of each of the feature points 401 constituting the input time-series feature points, and calculates an error between the polynomial g and the Y-coordinate of each of the feature points 401 constituting the input time-series feature points. An absolute value error or a squared error is adoptable as the error. A Huber error which is resistive against an outlier may be adopted in place of the error. The Huber error results in the squared error in a case where a difference between the polynomial g and each of the feature points 401 is equal to or less than 1, and results in an error function to be an absolute value error in a case where the difference is more than “1”. Even when a photographing direction of the camera 4 and the walking direction of the user do not agree with each other, utilization of the difference between the polynomial g and each feature point 401 as the error leads to an achievement of analysis of the walk without influence of displacement in feature points attributed to the angle of the camera 4.

### Step S9

The decision part 24 compares the input time-series feature points normalized in step S6 and corrected in step S7 with normalized reference time-series feature points stored in the walk database 32.

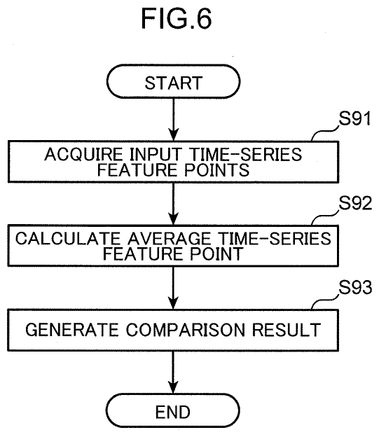

FIG. 6 is a flowchart showing details of step S9 in FIG. 3.

### Step S91

The decision part 24 acquires, from the calculation part 23, the normalized and corrected input time-series feature points. At this time, the decision part 24 stores the acquired input time-series feature points as reference time-series feature points in the walk database 32. In this manner, the walk database 32 stores the normalized and corrected input time-series feature points as the reference time-series feature points.

### Step S92

The decision part 24 calculates an average time-series feature point being a single time-series feature point by averaging a plurality of reference time-series feature points or first reference time-series feature points stored in the walk database 32. This enables one-to-one comparison of input time-series feature points with a plurality of reference time-series feature points. No calculation of the average time-series feature point leads to complicated one-to-two or more comparison of the input time-series feature points with the reference time-series feature points. Averaging of the reference time-series feature points is effective from the perspective of a reduction in noise involved in the reference time-series feature points. Specifically, the averaging provides an advantageous effect of reducing an estimation error in node estimation for the reference time-series feature points.

The decision part 24 may calculate the average time-series feature point by calculating an average value of feature points 401 for each of frames having the same number for each of the reference time-series feature points. In this case, when the number of frames, or the number of data pieces in a time direction, for time-series feature points differ from the number of frames for other time-series feature points, the decision part 24 may supplement feature points through linear interpolation to make the numbers agree with each other. For instance, in a case where an average time-series feature point is calculated from reference time-series feature points associated with ten frames and reference time-series feature points associated with thirty frames, no execution of interpolation leads to calculation of an average time-series feature point only for ten frames. This means that the twenty frames for associated reference time-series feature points remain without being used for calculation of the average time-series feature point. By contrast, execution of the interpolation enables calculation of an average time-series feature point for the thirty frames, resulting in achievement of more accurate calculation of the average time-series feature point.

### Step S93

The decision part 24 generates a comparison result by comparing the input time-series feature points normalized in step S6 and corrected in step S7 with the average time-series feature point obtained in step S92. Specifically, the decision part 24 may compare the input time-series feature points with the average time-series feature point by calculating feature point distances between the input time-series feature points and the average time-series feature point.

Each feature point distance is calculated as follows. First, the decision part 24 extracts feature points 401 concerning a predetermined number of frames respectively from the input time-series feature points and the reference time-series feature points. The predetermined number of frames may be an appropriate number of frames, for example, all the frames, or ten frames. The decision part 24 may calculate a distance between feature points 401 for input time-series feature points and calculate a distance between feature points 401 for average time-series feature points for every frame, and calculate a value resulting from averaging the calculated distances over all the frames to be a feature point distance.

The decision part 24 may compare the feature point distance with a threshold (e.g., a distance corresponding to twenty pixels), and generate a comparison result showing “no occurrence of a change in the walk” when the feature point distance is a distance of the threshold or shorter, or generate a comparison result showing “an occurrence of a change in the walk” when the feature point distance exceeds the distance of the threshold. Alternatively, the decision part 24 may compare a time-series feature amount calculated from the feature point 401 with a threshold, and generate a comparison result. For instance, the decision part 24 may calculate a distance between feature points 401 for input time-series feature points and a distance between feature points 401 for average time-series feature points for every frame, calculate differences between the calculated distances in adjacent frames, and calculate a value resulting from averaging the calculated differences over all the frames to be a time-series feature amount. Alternatively, the decision part 24 may calculate a distance between feature points 401 for input time-series feature points and a distance between feature points 401 for average time-series feature points for every frame, and calculate a statistical value of the calculated distances to be a time-series feature amount. The statistical value is, for example, an absolute value average, a root mean square value, a reference deviation, a distribution, or a distortion. The decision part 24 generates a comparison result showing an occurrence of a change or no occurrence of a change in the walk, but may generate a comparison result showing a feature point distance or a time-series feature amount in place of or in addition to a change or no change in the walk. Alternatively, a target user or an operator may compare a feature point distance or a time-series feature amount with a threshold through visual confirmation and input the comparison result into a terminal device carried by the target user or the operator to generate the comparison result.

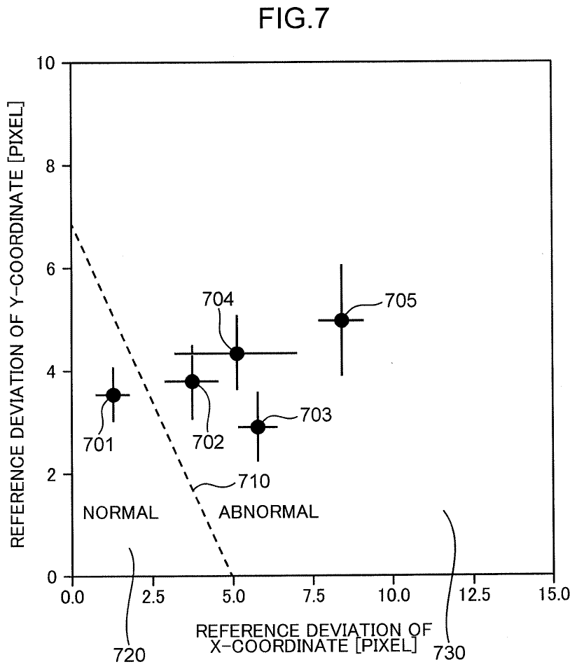

Further alternatively, the decision part 24 may decide a change in the walk by calculating a reference deviation of the error calculated in step S8 as the time-series feature amount and comparing the calculated time-series feature amount with a threshold straight line 710 denoted by a dotted-line in FIG. 7.

FIG. 7 is a graph explaining a way of deciding a change in the walk by using the reference deviation of the error calculated in step S8. In FIG. 7, the vertical axis denotes a reference deviation on a Y-coordinate, and the horizontal axis denotes a reference deviation on an X-coordinate. The reference deviation on the X-coordinate means a reference deviation about an error between a calculation value before correction on the X-coordinate and a polynomial g on the X-coordinate for one time-series feature point as calculated in step S8. The reference deviation on the Y-coordinate means a reference deviation about an error between a calculation value before correction on the Y-coordinate and a polynomial g on the Y-coordinate for one time-series feature point as calculated in step S8.

In FIG. 7, a point 701 shows a trial result about a normal uniform straight walk (straight), and points 702 to 705 show trial results about walks respectively imitating four kinds of abnormal walks. The point 702 shows a walk at a walking velocity which gradually increases or boosts, the point 703 shows a slow walk, the point 704 shows an awkward walk like stumbling, and the point 705 shows a habitual walk with a lowered right shoulder.

The graph in FIG. 7 shows a reference deviation of an error on the X-coordinate and a reference deviation of an error on the Y-coordinate in each of the five trials respectively for the five kinds of walks. Each of the points 701 to 705 resulting from plotting an average value of reference deviations on X-coordinates and an average value of reference deviations on Y-coordinates in the trials. Concerning each of the points 701 to 705, a vertical line segment indicates an error bar of a reference deviation on the Y-coordinate, and a horizontal line segment indicates an error bar of the reference deviation on the X-coordinate.

The threshold straight line 710 extends downward as advancing rightward and is defined to distinguish an abnormal walk from a normal walk by comparing the coordinate of the point 701 with the coordinate of the point 704 shown in FIG. 7. A region 720 located to the left of the threshold straight line 710 includes the point 701 indicating the normal walk, and thus is a region for decision of the normal walk. A region 730 located to the right of the threshold straight line 710 includes the points 702 to 705 each indicating an abnormal walk, and thus is a region for decision of the abnormal walk.

For instance, when the reference deviation of the error calculated in step S8 about each input time-series feature point determined to be in the walk period in step S5 falls within the region 720, the decision part 24 decides the walk as a normal walk. By contrast, when the reference deviation of the error obtained in step S8 about each input time-series feature point determined to be in the walk period in step S5 falls within the region 730, the decision part 24 decides the walk as an abnormal walk.

In employment of the way in FIG. 7, the memory 3 may store the threshold straight line 710 in advance, and the decision part 24 may read out the threshold straight line 710 as a reference time-series feature point from the memory 3 and decide a change in a walk by comparing the threshold straight line 710 with a reference deviation of an error of an input time-series feature point.

### Step S10

The output part 25 outputs the comparison result generated in step S93. The comparison result is output to, for example, a terminal device of the target user. As a result, when a change in the walk is decided, the terminal device displays a message indicating the change in an action of the walk on a display, or outputs the message from a speaker.

As described heretofore, in the first embodiment, the gravity center on node coordinates P belonging to the trunk is adopted as the feature point 401. Each node coordinate P belonging to the trunk gives a higher estimation accuracy than a node coordinate belonging to other part which is likely to move more frequently, such as a fingertip. Thus, each node coordinate P of the trunk can be stably estimated even in an action like a walking motion. Conclusively, the first embodiment achieves decision on a change in a walk of a target user with a high accuracy even in a case where the camera 4 is arranged in a residence at a camera angle which is unsuitable for sensing.

### Second Embodiment

The first embodiment does not consider whether a target user being photographed walks straight. In a case where the target user being photographed changes a walking direction, an accuracy of deciding a change in the walk may decrease. In this regard, it is determined in a second embodiment whether a target user being photographed walks straight.

Hereinafter, only the differences from the first embodiment will be described.

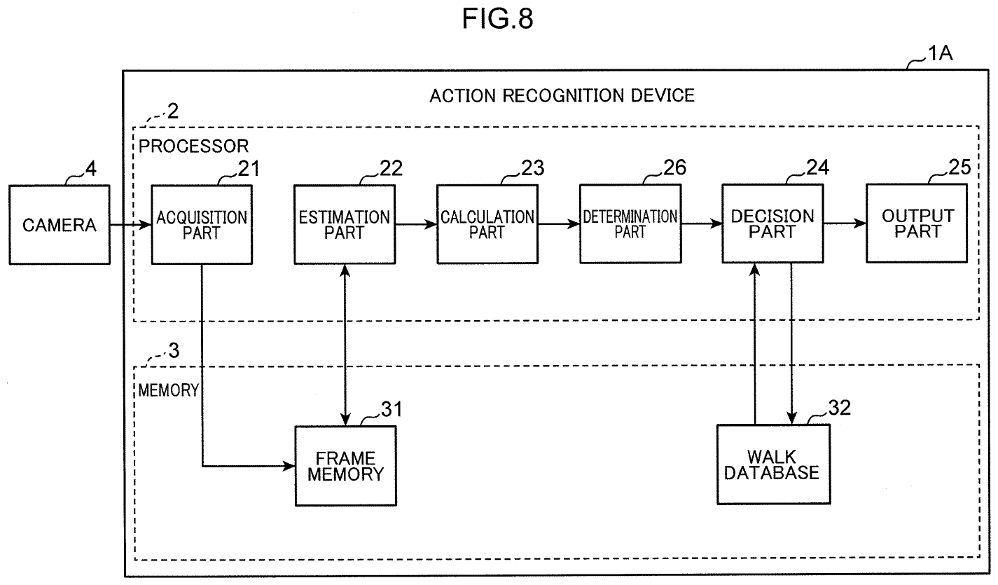

FIG. 8 is a block diagram showing an example of a configuration of an action recognition system in the second embodiment of the present disclosure.

In the second embodiment, elements which are the same as those in the first embodiment are given the same reference signs and numerals, and thus, description therefor will be omitted.

An action recognition device 1A further includes a determination part 26. The determination part 26 determines, on the basis of input time-series feature points calculated by a calculation part 23, whether a target user walks straight. A decision part 24 decides a change in a walk of the target user when the determination part 26 determines that the target user walks straight.

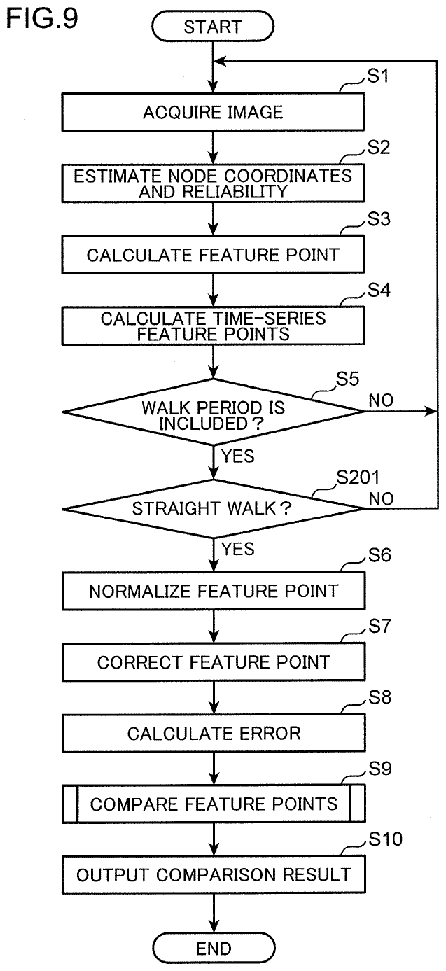

FIG. 9 is a flowchart showing an example of a process by the action recognition device 1A in the second embodiment of the disclosure. In FIG. 9, steps which are the same as those in FIG. 3 are given the same reference numerals and signs, and thus, description therefor will be omitted.

### Step S201

The determination part 26 determines whether a target user walks straight toward a camera 4. The determination part 26 calculates, on the basis of the following Formula (1), a trunk index for determining whether the front of the target user faces in the direction of the camera 4 per frame.

$$Trunk index = ( X - coordinate of right shoulder + X - coordinate of left waist ) - ( X - coordinate of right shoulder + X - coordinate of right waist ) \qquad ( 1 )$$

When the user walks straight toward the camera 4 and the front of the target user is photographed, the trunk index always has a positive value. By contrast, when the target user turns or turns back in the walking and the back of the target user is photographed, the trunk index has a negative value. Such determination of a positive value or a negative value of the trunk index leads to achieved determination as to whether the front of the target user faces in the direction of the camera 4.

The determination part 26 determines whether a proportion of the number of frames in which the trunk index has positive values to the total frames in a walking period indicates a threshold or higher. The threshold is, for example, 0.7. The determination part 26 determines that the target user walks straight toward the camera 4 when the proportion of the number of frames in which the trunk index has positive values to the total frames in the walking period indicates the threshold or higher. By contrast, the determination part 26 determines that the target user does not walk straight toward the camera 4 when the proportion of the number of frames in which the trunk index has positive values to the total frames in the walking period indicates a value lower than the threshold.

When the target user is determined not to walk straight toward the camera 4 (NO in step S201), the process returns to step S1.

By contrast, when the target user is determined to walk straight toward the camera 4 (YES in step S201), the process proceeds to step S6.

The second embodiment achieves recognition of an action of a target user with a high accuracy by removing a feature point resulting from calculation not based on walking straight but based on walking in a different direction that gives an adverse influence on the recognition of a change in a walking motion.

### Third Embodiment

A third embodiment is aimed at correcting a feature point 401 in relation to a distance from a camera 4 to a target user. A size of the target user shown in an image depends on a depth (a horizontal distance) from the target user to a location of the camera 4. When the target user is defined to substantially walk in a direction of the camera, a size of the target user shown in the image is proportional to an inverse of the depth. That is to say, the target user is seen larger as the target user comes closer to the camera 4. In the third embodiment, depth correction to be described later is hence executed to exclude a change in the size of the target user attributed to a distance from the camera 4.

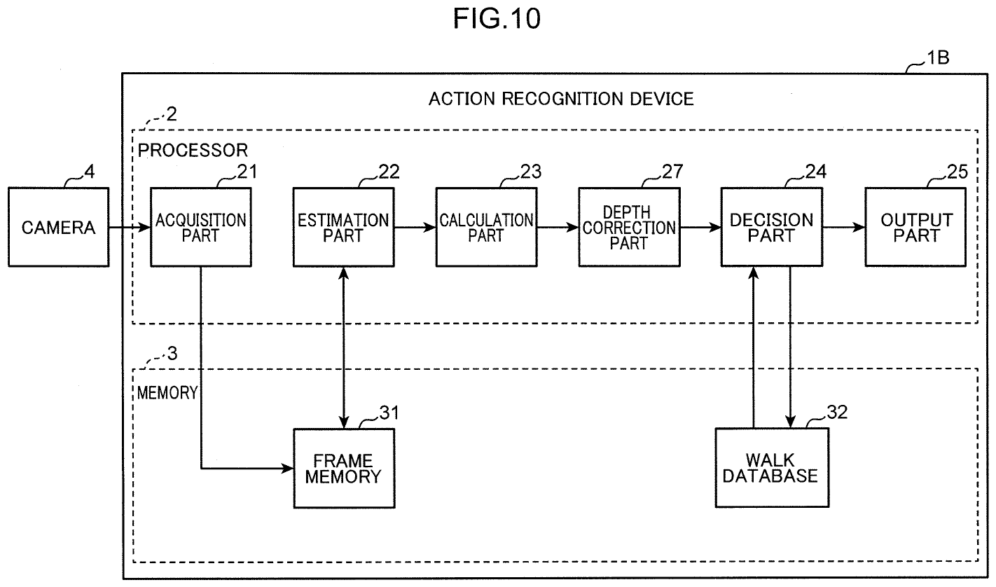

FIG. 10 is a block diagram showing an example of a configuration of an action recognition system in the third embodiment of the present disclosure.

In the third embodiment, elements which are the same as those in the first embodiment are given the same reference signs and numerals, and thus, description therefor will be omitted.

An action recognition device 1B further includes a depth correction part 27. The depth correction part 27 calculates correction factors to distances between a target user shown in an image acquired by an acquisition part 21 and a camera that captures images of the target user in time series. Here, the correction factors represent a plurality of correction factors for respective frames for input time-series feature points. Each correction factor has a value increasing as a distance between the target user shown in the image to a camera 4 is longer.

A decision part 24 decides a change in a walk of the target user by normalizing time series feature points by multiplying feature points constituting the time series feature points by correction factors corresponding to the feature points respectively, and comparing the normalized time-series feature points with reference time-series feature points which are normalized.

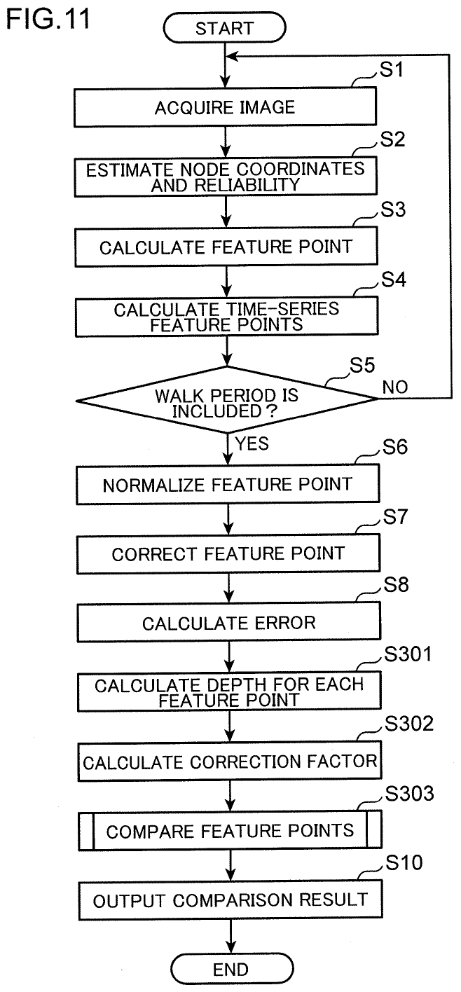

FIG. 11 is a flowchart showing an example of a process by the action recognition device 1B in the third embodiment of the disclosure. In FIG. 11, steps which are the same as those in FIG. 3 are given the same reference numerals and signs, and thus, description therefor will be omitted.

### Step S301

The depth correction part 27 calculates a depth for each feature point constituting input time-series data. Specifically, the depth correction part 27 acquires, from a memory 3, a value defined in advance as a walking distance of the target user in a space around the camera 4. The walking distance is a distance from a walking start position at which the target user appears in a frame of the camera 4 to a walking finish position at which the target user goes out of the frame of the camera 4 in the space around the camera 4. The walking distance may take a value input by the user via a terminal device, or may take a value of a general walking distance to and from an entrance hall (e.g., 1.9 mm). The depth correction part 27 calculates a depth from the camera 4 to the user in frames respectively associated with input time-series feature points on the premise that the target user is in a linear motion between the walking start position and the walking finish position.

For instance, the depth correction part 27 defines the location of the camera 4 as a start position and divides a walking distance by total frames required for a walk to obtain a walking distance per frame. The depth correction part 27 is only required to obtain the depth for each frame by multiplying the number of frames from a frame in which the user appears to the relevant frame by a walking distance for each frame.

### Step S302

The depth correction part 27 calculates a correction factor for each frame from a depth in each frame calculated in step S301.

Specifically, the depth correction part 27 may calculate a correction factor by using the following Formula (2).

$$wi = di / W , however , W = {\sum }_{i} = {}_{1}^{M} {w}_{i} \qquad ( 2 )$$

The sign “wi” denotes a correction factor of the i-th frame, and the sign “di” denotes a depth of the i-th frame. The sign “M” denotes the number of frames concerning the input time-series feature points, and the sign “W” denotes a sum of correction factors wi. Specifically, the correction factor wi is normalized in such a manner that a total sum results in 1.

### Step S303

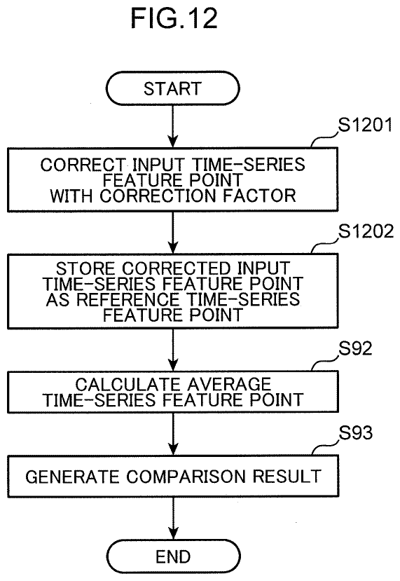

Step S303 is shown in a flowchart of FIG. 12. FIG. 12 is the flowchart showing details of step S303 in FIG. 11. In FIG. 12, steps which are the same as those in FIG. 6 are given the same reference numerals and signs, and thus, description therefor will be omitted.

### Step S1201

A decision part 24 corrects each feature point 401 included in input time-series feature points corrected in step S7 by multiplying the correction factor wi calculated in step S302 by the feature point 401.

### Step S1202

The decision part 24 stores, in the walk database 32, each input time-series feature point resulting from correcting each feature point 401 by the correction factor wi in step S1201 as a reference time-series feature point.

Hereinafter, steps S92 to S93 are executed to decide a change in the walk of the user. The walk database 32 stores the input time-series feature point resulting from correcting each feature point 401 by the correction factor wi in step S1202 as a reference time-series feature point. That is, an average time-series feature point calculated in step S92 results from correcting each feature points 401 by the correction factor wi. Hence, in step S93, a comparison result is generated by comparing the input time-series feature point resulting from correcting each feature point 401 by the correction factor wi with the average time-series feature point resulting from correcting the feature point 401 by the correction factor wi.

Conclusively, the third embodiment enables decision on a change in a walk of a target user after excluding a change in a size of the target user shown in an image attributed to a depth from the camera 4 that gives an adverse influence on the decision on the change in a walking motion, and thus achieves decision on a change in the walk of the user with a high accuracy.

### Modifications

Although an action recognition device according to one or more aspects of the present disclosure on the basis of embodiments has been described heretofore, the present invention is not limited to the embodiments. Various modifications applied to the embodiments or configurations obtained by combining constituent elements in the embodiments different from each other conceived by a person skilled in the art may be included in the scope of more features of the present disclosure as far as such modifications and configurations do not deviate from the gist of the disclosure.
(1) The second embodiment and the third embodiment may be combined. Specifically, the depth correction part 27 may calculate the correction factor wi when the determination part 26 determines the straight walk.
(2) The output part 25 outputs a message indicating a change in a walk, but this is just an example, and may output a message indicating a decline in the walk. Alternatively, the output part 5 may output an index indicating a change in the walk. Examples of the index may include a feature point distance or a time-series feature amount as described above.

An action recognition device according to this disclosure is useful for recognizing a state of a user in a residence.

1. An action recognition device that recognizes a walk of a user, comprising:
an acquisition part that acquires an image;
an estimation part that estimates node coordinates of the user from the image acquired by the acquisition part;
a calculation part that calculates, on the basis of the node coordinates estimated by the estimation part, time-series feature points each indicating a feature point of a trunk of the user in time series;
a storage part that stores time-series feature points being reference time-series feature points each to be a reference; and
a decision part that decides a change in the walk of the user by comparing input time-series feature points being the time-series feature points calculated by the calculation part with the reference time-series feature points.

2. The action recognition device according to claim 1, wherein the time-series feature points are normalized in such a manner that a time required for the walk is a reference value.

3. The action recognition device according to claim 1, further comprising a determination part that determines, on the basis of the time-series feature points calculated by the calculation part, whether the user walks straight, wherein
the decision part decides a change in the walk of the user when the user is determined to walk straight.

4. The action recognition device according to claim 1, further comprising a depth correction part that calculates correction factors to distances between the user shown in the image and a camera that captures the image of the user in time series, wherein
the decision part decides a change in the walk of the user by normalizing the time-series feature points by multiplying feature points constituting the time series feature points by correction factors corresponding to the feature points respectively, and comparing the normalized time-series feature points with the reference time-series feature points which are normalized.

5. The action recognition device according to claim 1, wherein the calculation part calculates respective polynomial approximate curves of X-coordinates and Y-coordinates of the time-series feature points on the basis of image coordinates of the calculated time-series feature points, and corrects respective values of X-coordinates and Y-coordinates of feature points constituting the time-series feature points by using the calculated polynomial approximate curves of the X-coordinates and the Y-coordinates.

6. The action recognition device according to claim 1, wherein the reference time-series feature points include a plurality of first reference time-series feature points, and
the decision part decides a change in the walk of the user by calculating an average time-series feature point by averaging the first reference time-series feature points, and comparing the average time-series feature point with the input time-series feature points.

7. The action recognition device according to claim 1, wherein,
in a case where a period during which time-series feature points are continuously calculated is not shorter than a predetermined time, the calculation part determines the feature points within the period as the input time-series feature points.

8. The action recognition device according to claim 1, wherein the reference time-series feature points represent input time-series feature points calculated by the calculation part in past.

9. The action recognition device according to claim 1, wherein the reference time-series feature points and the input time-series feature points belong to the same user.

10. The action recognition device according to claim 1, further comprising an output part that outputs information indicating the change in the walk of the user decided by the decision part.

11. An action recognition method for an action recognition device that recognizes a walk of a user, comprising:
acquiring an image;
estimating node coordinates of the user from the acquired image;
calculating, on the basis of the estimated node coordinates, time-series feature points each indicating a feature point of a trunk of the user; and
deciding a change in the walk of the user by comparing input time-series feature points being the calculated time-series feature points with time-series feature points being reference time-series feature points each to be a reference.

12. A non-transitory computer readable recording medium storing an action recognition program for causing a computer to execute an action recognition method for recognizing a walk of a user, comprising:
causing the computer to execute:
acquiring an image;
estimating node coordinates of the user from the acquired image;
calculating, on the basis of the estimated node coordinates, time-series feature points each indicating a feature point of a trunk of the user; and
deciding a change in the walk of the user by comparing input time-series feature points being the calculated time-series feature points with time-series feature points being reference time-series feature points each to be a reference.
